(19) United States
(12) Patent Application Publication
(10) Pub. No.: US 2022/0251671 A1
(43) Pub. Date: Aug. 11, 2022

(54) SYSTEMS AND METHODS FOR DETECTING THE PRESENCE OF AN ANALYTE, SUCH AS SARS-COV-2, IN A SAMPLE

(71) Applicant: ADL Diagnostics, Woodinville, WA (US)

(72) Inventor: Robert G. ATKINSON, Woodinville, WA (US)

(73) Assignee: ADL Diagnostics, Woodinville, WA (US)

(21) Appl. No.: 17/666,338

(22) Filed: Feb. 7, 2022

Related U.S. Application Data
(60) Provisional application No. 63/146,259, filed on Feb. 5, 2021.

(51) Int. Cl. C12Q 1/70; C12Q 1/6844; C12Q 1/6888; C12N 9/22

(52) U.S. Cl. CPC C12Q 1/701; C12N 9/22; C12Q 1/6888; C12Q 1/6844

(57) ABSTRACT

Methods for detecting an analyte in a sample are disclosed. The method can include depositing the sample in an instrument, such as a Loop-Mediated Isothermal Amplification (LAMP) instrument that is configured to selectively amplify an analyte, such as a characteristic portion of a genome of a pathogen. A moving average of the quantity of the analyte at an instance of time can be compared to a sum of (1) the moving average for a previous instance of time and (2) a multiple of the moving standard deviation at the previous instance of time. If the quantity of the analyte at the instance of time is greater than the sum of (1) the moving average for a previous instance of time and (2) a multiple of the moving standard deviation at the previous instance of time, it can be an indication that the sample is positive for the analyte.

### CROSS REFERENCE TO RELATED APPLICATION

This application claims priority to and the benefit of U.S. Provisional Patent Application No. 63/146,259, the entire disclosure of which is hereby incorporated by reference

### FIELD

Embodiments described herein generally relate to systems and methods for selectively amplifying a target analyte and determining whether the target analyte is present or absent in a sample based on a change of a signal associated with the quantity of the analyte. Some embodiments described herein are particularly suitable for diagnostic tests configured to determine whether a sample taken from a patient contains severe acute respiratory syndrome coronavirus 2 (SARS-CoV-2), the pathogen responsible for the global COVID-19 pandemic.

### BACKGROUND

A number of diagnostic and analytic techniques have been developed to detect the presence of proteins, DNA, or other suitable biomarkers, for example, those associated with SARS-CoV-2. Many such techniques are designed to amplify a target analyte for a predetermined period of time and then determine whether a quantity of the target analyte is detectable and/or exceeds a predetermined threshold that indicates a “positive” result. Such techniques are time consuming, as they must generally be run to completion, or at least until a signal associated with the target analyte crosses a pre-defined threshold. The lengthy run time for such techniques has contributed to significant delays in obtaining test results. For example, in many cases, the wait time to obtain a COVID-19 test result is 7-10 days. A need therefore exists for systems and methods capable of reducing the run time necessary to determine the presence of an analyte in a sample. Systems and methods described herein are well suited for “rapid” testing, potentially producing results in under half an hour and while the patient waits, which can significantly contribute to curbing the spread of COVID-19.

### SUMMARY OF THE INVENTION

Some embodiments described herein relate to a method for detecting the presence of an analyte in a sample. The method can include depositing the sample in an instrument, such as a Loop-Mediated Isothermal Amplification (LAMP) instrument that is configured to selectively amplify an analyte, such as a characteristic portion of a genome of a pathogen. A quantity of the analyte can be continuously monitored by receiving a signal, such as a fluorescent signal, associated with the analyte. A moving average of the quantity of the analyte and a moving standard deviation of the quantity of the analyte can be calculated. The moving average of the quantity of the analyte at an instance of time can be compared to a sum of (1) the moving average for a previous instance of time and (2) a multiple of the moving standard deviation at the previous instance of time. If the quantity of the analyte at the instance of time is greater than the sum of (1) the moving average for a previous instance of time and (2) a multiple of the moving standard deviation at the previous instance of time, it can be an indication that the sample is positive for the analyte.

Some embodiments described herein relate to a system for evaluating a sample to determine whether it contains an analyte. The system can include a well configured to receive a reaction tube containing an analyte, a light emitting source configured to emit an excitation light at a wavelength to illuminate the analyte in the reaction tube, an optical detector configured to receive optical signals in response to the analyte being illuminated by the excitation light, and a processor operably coupled to the light emitting source and the optical detector. The processor can be configured to activate the light emitting source, receive a plurality of signals from the optical detector, each signal from the plurality of signals associated with the optical signals and indicative of a quantity of an analyte at an instance of time, calculate, for each instance of time, a moving average of the quantity of the analyte and a moving standard deviation of the quantity of the analyte based on a subset of the plurality of signals associated with a period of time ending at that instance of time, and compare the moving average of the quantity of the analyte at a first instance of time with a sum of (1) the moving average for a second instance of time and (2) a multiple of the moving standard deviation at the second instance of time, the second instance of time being an amount of time before the first instance of time.

Some embodiments described herein relate to a computer implemented method (e.g., a non-transitory computer-readable medium storing instructions configured to cause a processor to perform a method). The computer implemented method can include receiving a plurality of signals, each signal from the plurality of signals associated with a quantity of an analyte at an instance of time. A moving average and a moving standard deviation of the quantity of the analyte can be calculated for each instance of time based on a subset of the plurality of signals associated with a period of time ending at that instance of time. The moving average of the quantity of the analyte at a first instance of time can be compared to a sum of (1) the moving average for a second instance of time and (2) a multiple of the moving standard deviation at the second instance of time, the second instance of time being an amount of time before the first instance of time. A signal indicating a positive result can be generated and/or sent based on the moving average of the quantity of the analyte at the first instance of time being greater than the sum of (1) the moving average for the second instance of time and (2) a multiple of the moving standard deviation at the second instance of time.

Some embodiments described herein relate to a method of determining the presence or absence of an analyte is a biological sample. The method can include depositing a biological sample in an instrument configured to selectively amplify an analyte. A plurality of signals can be received—each signal from the plurality of signals can be associated with a quantity of an analyte at an instance of time. A moving average of the quantity of the analyte and a moving standard deviation of the quantity of the analyte can be calculated for each instance of time based on a subset of the plurality of signals associated with a period of time ending at that instance of time. The moving average of the quantity of the analyte at a first instance of time can be compared to a sum of (1) the moving average for a second instance of time and (2) a multiple of the moving standard deviation at the second instance of time, the second instance of time being an amount of time before the first instance of time. When the moving average of the quantity of the analyte at the first instance of time is greater than the sum of (1) the moving average for the second instance of time and (2) a multiple of the moving standard deviation at the second instance of time, the biological sample can be determined to contain the analyte in a greater than a threshold quantity. When (a) the moving average of the quantity of the control at the first instance of time is greater than the sum of (1) the moving average of the quantity of the control for the second instance of time and (2) a multiple of the moving standard deviation of the quantity of the control at the second instance of time and (b) the moving average of the quantity of the analyte at the first instance of time is less than the sum of (1) the moving average of the quantity of the analyte for the second instance of time and (2) a multiple of the moving standard deviation of the quantity of the analyte at the second instance of time, the biological sample can be determined to contain less than a threshold quantity of the analyte when.

### BRIEF DESCRIPTION OF THE FIGURES

FIGS. 1A-1D depict an instrument operable to amplify an analyte and measure a signal associated with a quantity of the analyte, according to an embodiment.

FIG. 2 is a flow chart of a method of detecting an analyte, according to an embodiment.

FIG. 3 is an experimental data from a FLOS-LAMP analysis of an example sample.

### DEFINITIONS

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “sample” refers to a composition that contains an analyte or analytes. A sample can be heterogeneous, containing a variety of components or homogenous, containing one component. In some instances, a sample can be naturally occurring, a biological material, and/or a man-made material. Furthermore, a sample can be in a native or denatured form.

In certain embodiments, the sample is a biological sample. In some instances, a sample can be a single cell (or contents of a single cell) or multiple cells (or contents of multiple cells), a saliva sample, a mucous sample, a blood sample, a tissue sample, a skin sample, a urine sample, a water sample, and/or a soil sample. In some instances, a sample can be from a living organism, such as a eukaryote, prokaryote, mammal, human, yeast, and/or bacterium or the sample can be from a virus. In some embodiments, a sample can be a food product or a beverage product. In some embodiments, a sample can be a swab of a surface, e.g., a swab of a food preparation surface or a container. Biological samples include, but are not limited to, tissues, cells and biological fluids obtained from a subject. For example, biological samples include, but are not limited to, blood and a fraction or component of blood including blood serum, blood plasma, or lymph, saliva, nasal fluid, etc. In certain embodiments, the biological sample is a blood sample, a serum sample, a saliva sample, a mucous sample, a tissue sample, a skin sample, a urine sample. In one embodiment, the biological sample contains virus or protein molecules from the test subject. The biological sample may be a peripheral blood leukocyte sample isolated by conventional means from a subject. In certain embodiments, the biological sample is selected from the group consisting of: serum, blood, salivary secretions (e saliva), lacrimal secretions (e.g, tears), respiratory secretions (e.g., mucus), nasal fluid, a nasal swab, an oral swab, a mucous sample, and intestinal secretions mucus).

As used herein, the term “analyte” refers to any molecule or compound to be detected as described herein. Suitable analytes can include but are not limited to, small chemical molecules and/or biomolecules, such as, for example, environmental molecules, clinical molecules, chemicals, and pollutants. More specifically, such chemical molecules and/or biomolecules can include but are not limited to pesticides, insecticides, toxins, therapeutic and/or abused drugs, hormones, antibiotics, antibodies, organic materials, proteins (e.g., enzymes, immunoglobulins, and/or glycoproteins), nucleic acids (e.g., DNA and/or RNA), lipids, lectins, carbohydrates, whole cells (e.g., prokaryotic cells such as pathogenic bacteria and/or eukaryotic cells such as mammalian tumor cells), viruses, spores, polysaccharides, glycoproteins, metabolites, cofactors, nucleotides, polynucleotides, transition state analogs, inhibitors, nutrients, electrolytes, growth factors and other biomolecules and/or non-biomolecules, as well as fragments and combinations thereof. Some analytes described herein can be proteins such as enzymes, drugs, cells, antibodies, antigens, cellular membrane antigens, and/or receptors or their ligands (e.g., neural receptors or their ligands, hormonal receptors or their ligands, nutrient receptors or their ligands, and/or cell surface receptors or their ligands). In particular embodiments, an analyte is an infectious or pathological agent, such as, e.g., a bacterium, virus, yeast, or fungus.

As used herein, the term “protein” refers to proteins, polypeptides, oligopeptides, peptides, and analogs, including proteins containing non-naturally occurring amino acids and amino acid analogs, and peptidomimetic structures. The term “protein” also refers to proteins, polypeptides, oligopeptides, peptides, and analogs.

### DETAILED DESCRIPTION

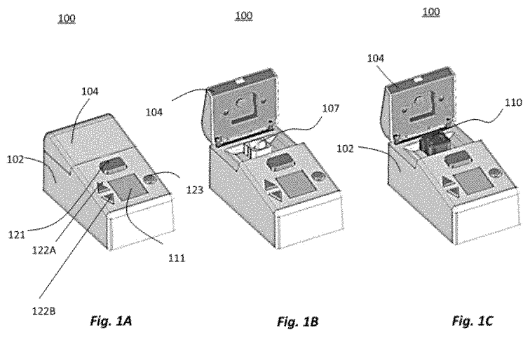

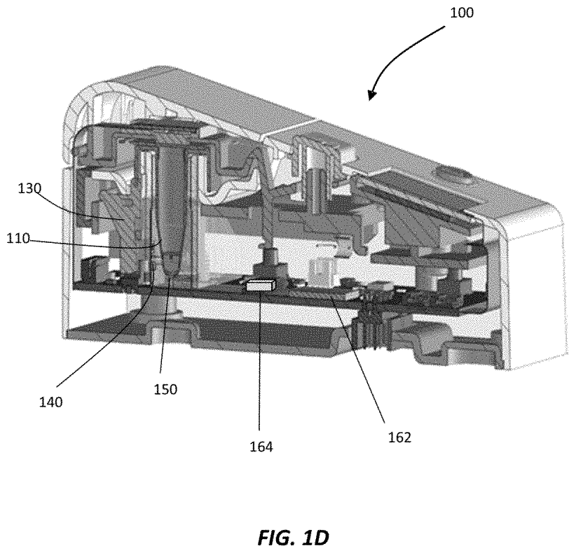

FIGS. 1A-1C depict a FLOS-LAMP (Fluorescence of Loop Primer Upon Self Dequenching Loop-Mediated Isothermal Amplification) instrument 100 operable to amplify an analyte and measure a signal associated with a quantity of the analyte, according to an embodiment. FIG. 1B depicts the instrument 100 in an open and empty configuration. FIG. 1C shows a reaction tube 110 containing a sample is disposed in the instrument 100. FIG. 1A depicts the instrument 100 in a closed configuration. With the cover 104 closed, the instrument can be configured to selectively amplify polynucleotide sequence(s). For example, the reaction tube 110 can include suitable primers to selectively cause one or more analytes and/or one or more controls in the sample to be amplified according to known techniques (e.g., Loop-Mediated Isothermal Amplification).

Instrument 100 includes a housing 102 configured to receive reaction tube 110. Cover 104 can be coupled to housing 102, as shown in FIG. 1A. In an example embodiment, cover 104 may be a hinged cover (or any other suitable cover attached in any other suitable way to instrument 100) configured to be movable to cover a top portion of reaction tube 110. In some cases, cover 104 is configured to contain (and, in some cases, lock) the reaction tube 110 into the housing 102.

FIG. 1A also shows a display screen 111 configured to display information during and after performance of the assay. Further instrument 100 may have a cover button 121 for opening cover 104. Further, there may be a select button for selecting options displayed on screen 111, and up and down respective buttons 122A and 122B for moving up or down between options on the screen. In various embodiments, the options may be associated with a type of assay that is being performed.

Underneath cover 104, housing 102 may include a well 107 for placing reaction tube 110, as shown in FIG. 1B. FIG. 1C shows rection tube 110 placed in well 107 of section 102. In various embodiments, instrument 100 is configured to amplify an analyte using Polymer Chain Reaction (PCR), Loop-Mediated Isothermal Amplification (LAMP), Real-Time FLOS (RT-LAMP), Fluorescence of Loop primer upon self-dequenching (FLOS LAMP, or any other suitable techniques. Instrument 100 can further be configured to measure a signal associated with a quantity of the analyte, according to an embodiment. For example, instrument 100 may be configured to selectively amplify polynucleotide sequence(s). For example, the reaction tube 110 can include suitable primers to selectively cause one or more analytes and/or one or more controls in the sample to be amplified according to known techniques (e.g., LAMP, etc.).

In various embodiments, reaction tube 110 includes a body portion closed at a bottom portion, the bottom portion being at least partially transparent to excitation light at an excitation wavelength and to emission light at an emitted wavelength.

FIG. 1D is a cross sectional view of the instrument 100 in the closed configuration with the reaction tube 110 disposed within. A heating block 130 is configured to control the temperature of the reaction tube and sample, for example, to maintain the temperature of analytes within the sample (containing appropriate primers) to cause the analytes to undergo isothermal amplification. The heating block 130 includes a hole that provides an optical pathway such that a light emitting source 140 (e.g., light emitting diodes, lasers, etc.) can illuminate the sample at a predetermined wavelength and/or excite fluorescent dyes contained within the reaction tube 110. Another hole through the heating block 130 provides an optical pathway such that a sensor 150 (e.g., photodiodes, photomultipliers, charge coupled devices (CCDs) and/or any other suitable optical detectors) can detect optical signals, such as fluorescent signals emitted by fluorescent dyes and associated with a quantity and/or concentration of analyte(s) and/or control(s). In other embodiments, the instrument 100 can contain any other suitable sensor operable to detect signals characteristic of quantity and/or concentration of analytes and/or controls, such as optical sensors configured to detect native fluorescence, absorbance, and/or color (change), electrochemical sensors, pH sensors, or any other sensor operable to detect a signal indicative of a concentration and/or quantity of an analyte.

The instrument includes a processor 162 and/or a memory 164. The processor 162 can be, for example, a general purpose processor, a Field Programmable Gate Array (FPGA), an Application Specific Integrated Circuit (ASIC), a Digital Signal Processor (DSP), and/or the like. The processor 162 can be configured to retrieve data from and/or write data to memory, e.g., the memory 164, which can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth.

The processor 162 and the memory 164 can be communicatively coupled to the heating block 130, light source 140 and/or sensor 150 and configured to control a run during which analytes and/or controls disposed within the reaction tube 110 are selectively amplified. The processor and the memory can be operable to receive, process, and/or record signals associated with concentrations of analytes and/or controls. The processor and/or memory can be configured to determine whether the sample contained within the reaction tube 110 is “positive” or “negative” for one or more analytes, according to methods described in further detail herein. Although shown within a housing of instrument 110, in other embodiments, the processor 162 and/or memory 164 can be disposed in another device. Similarly stated, instrument 110 can be communicatively coupled to an external compute device configured to control a run and/or determine whether the sample is positive or negative.

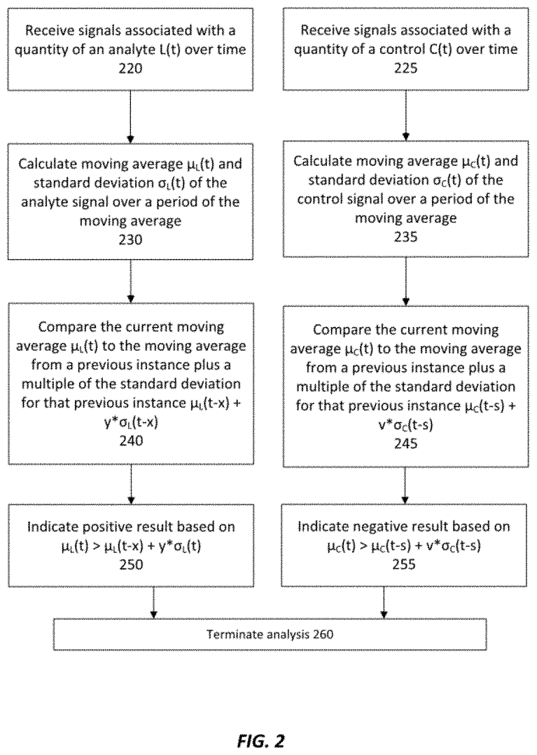

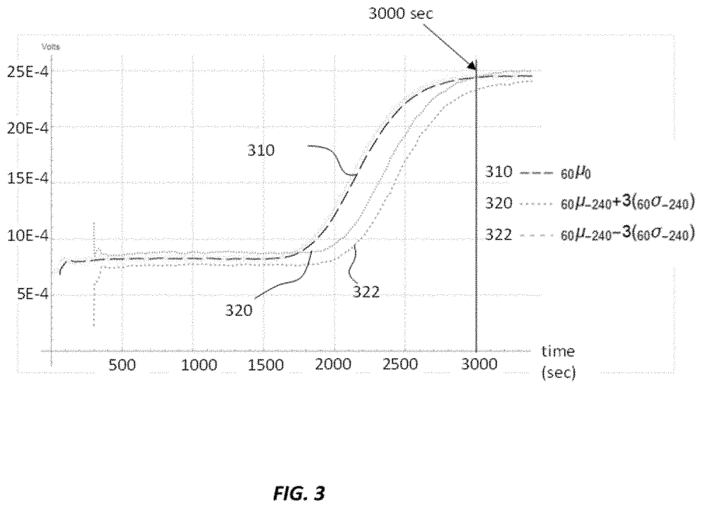

FIG. 2 is a flow chart of a method of detecting an analyte, according to an embodiment. The method shown and described in FIG. 2 can be performed by the instrument 100 shown and described with reference to FIG. 1 or any other suitable instrument configured to selectively amplify an analyte. Throughout the method, the instrument can analyze a sample over time. For example, the instrument can be configured to perform LAMP to amplify an analyte, such as a characteristic sequence of the SARS-CoV-2 genome. During the analysis, the instrument can continuously receive signal(s) associated with a quantity of the analyte, at 220. The instrument can be configured to receive a signal associated with a quantity of the analyte every second, every 5 seconds, every 10 seconds, every 20 seconds, or any other suitable sampling rate. For example, in FLOS-LAMP techniques, a labeled loop probe can be configured to fluoresce when bound to the analyte such that the intensity (L) of light emitted from the fluorophore label can be used to determine a quantity and/or concentration of the analyte as the sample is selectively amplified. The signals received at 220 can represent time-series data for the intensity of the fluorophore and/or concentration of the analyte.

The instrument (or a compute device coupled to the instrument) can be operable to process the signal(s) associated with the analyte that are received at 220. The instrument can calculate a moving average of the intensity (μL) and a standard deviation of the intensity (OL), at 230. Typically, the moving average and the standard deviation will have the same window. The width of the moving average and moving standard deviation windows can be predetermined and/or dynamic. For FLOS-LAMP signals, a suitable fixed window over which the moving average and/or standard deviation are calculated can be at least or about 3 minutes, at least or about 2 minutes, at least or about 60 seconds, at least or about 30 seconds, at least or about 20 seconds, or any suitable length of time. In some embodiments, the window over which the moving average and/or standard deviation are calculated can be a function of elapsed time and/or temperature of the amplification reaction such that, for example, the length of the window decreases as the run progresses.

In other embodiments, the instrument can further process the intensity measurement to calculate a quantity of the analyte. The moving average and moving standard deviation can then be calculated for the quantity of the analyte, rather than for the intensity of the fluorophore.

Calculating the moving average and the moving standard deviation produces a data set that can be stored in memory such that a data set includes, for each instance of time (t), an instantaneous intensity (L(t)), an average intensity over a period of time ending at the instance (μ_{L}(t)), and a standard deviation of intensity measurements taken over the period of time ending at that instance (σ_{L}(t)). Moving average and moving standard deviations can be calculated substantially in real time (e.g., within less than a second) as the intensity measurements are made. Once calculated, at 240, a moving average of the intensity can be compared to a sum of the moving average of the intensity calculated for a previous instance in time and a multiple of the standard deviation of the intensity calculated for that previous instance in time:

μ_{L}(t−x)+y*σ_{L}(t−x) (equation 1)
where x represents the difference in time between the current instance and the previous instance; and
y is a constant or function by which the moving standard deviation is multiplied.
   For FLOS-LAMP analyses, a suitable x is about 8 minutes, about 6 minutes, about 4 minutes, about 2 minutes, or any other suitable time. A suitable y is 1.2, 1.5, 1.8, 2, 2.5, 3, 4, or any other suitable value. Similarly stated, for a FLOS-LAMP analysis a current (e.g., most recently calculated) moving average of intensity can be compared to the moving average of the intensity calculated 4 minutes previously plus 2 times the standard deviation of intensity calculated 4 minutes previously. The current moving average of intensity being greater than the sum of the moving average of the intensity calculated for a previous instance in time and a multiple of the standard deviation of the intensity calculated for that previous instance in time

μ_{L}(t)>μ_{L}(t−x)+y*σ_{L}(t−x) (equation 2)

can be referred to as the target indicating. The target indicating can represent a positive result, or a presence of the analyte in the sample. In some instances, upon determining that a sample is positive, a signal indicating the positive result can be immediately (e.g., within 3 seconds) sent (e.g., to a user or technician), at 250, and/or the sample run can be terminated at 260. In other instances, the indication of a positive result can be sent based on the target indicating for a period of time (e.g., 5 seconds, 10 second, 15 seconds, 30 seconds, etc.). In this way, samples can be continuously be evaluated for positivity during amplification and the run can be terminated upon detecting a positive result, which can eliminate the need to amplify the sample for a predetermined period of time and evaluating the sample after processing. Such a technique can, in many instances, result in much shorter run times compared to known methods.

In some instances, a similar technique can be applied to a control signal to detect negative results (e.g., the absence of the analyte from the sample and/or the sample containing a quantity of the analyte that is below a detection threshold). The sample can contain one or more internal controls and a fluorescent tags configured to produce a luminescent signal indicative of a quantity of the controls. Typically, the sample will contain a control with a known initial quantity and/or concentration. The instrument can be configured to selectively amplify the control(s) simultaneously with the analytes such that, given a known initial quantity/concentration of a control, the time for that control to indicate can be predicted. As discussed in further detail herein the sequence and/or difference in time between the control indicating and the analyte indicating can be used to determine whether the sample is positive or negative. Therefore, the initial quantity/concentration of the control can be associated with a detection threshold of the analyte. The sample can also include additional controls configured to indicate if the sample run fails for various reasons. For example, a control can be used to determine whether a sufficient volume of sample was obtained. RNaseP, which is known to be present in predictable concentrations in human nasal mucous, can be used to evaluate whether a sufficient volume of human nasal mucous sample is present. The failure of an RNaseP control to indicate (e.g., before a control having a known concentration indicates) can therefore cause the instrument to send a signal indicating that the test was inconclusive for insufficient sample.

In some embodiments, the internal control may be endogenous to the sample, e.g., a biological sample, or the internal control may be added to the sample. In one non-limiting example, when detecting the presence of viral DNA or RNA in a biological sample, the internal control may be RNA expressed from a housekeeping gene or ribosomal RNA. Typically, the luminescent signal(s) indicative of the quantity of the control(s) will have a different spectral and/or temporal characteristic than the luminescent signal indicative of the quantity of the analyte. In other instances, the sample can be subdivided into two or more subsamples. Each subsample can be configured to be analyzed for one or more different analytes and/or serve as a control for one or more different analytes. In such an embodiment, each subsample would typically be amplified simultaneously. During the analysis of the sample, the instrument can continuously receive signal(s) associated with a quantity of the control, at 225.

The instrument (or the compute device coupled to the instrument) can be operable to process the signal(s) associated with the control that are received at 225. The instrument can calculate a moving average of the intensity of the control signal (μ_{C}) and a standard deviation of the intensity of the control signal (σ_{C}), at 235. In other embodiments, the instrument can further process the intensity measurement to calculate a quantity of the control. The moving average and moving standard deviation can then be calculated for the quantity of the control, rather than for an intensity associated with the quantity of the control.

Calculating the moving average and the moving standard deviation produces a data set that can be stored in memory such that a data set includes, for each instance of time (t), an instantaneous intensity of the control signal (C(t)), an average intensity of the control signal over a period of time ending at the instance (μ_{C}(t)), and a standard deviation of intensity of the control signal taken over the period of time ending at that instance (σ_{C}(t)). The current moving average of the intensity of the control signal being greater than a sum of the moving average of the intensity of the control signal calculated for a previous instance in time and a multiple of the standard deviation of the intensity of the control signal calculated for that previous instance in time

μ_{C}(t)>μ_{L}(t−s)+v*σ_{C}(t−s) (equation 3)
(where s represents the difference in time between the current instance and the previous instance; and
v is a constant or function by which the moving standard deviation is multiplied)
   can be referred to as the control indicating. For FLOS-LAMP analyses, a suitable s is about 8 minutes, about 6 minutes, about 4 minutes, about 2 minutes, or any other suitable time. A suitable v is 1.2, 1.5, 1.8, 2, 2.5, 3, 4 or any other suitable value. In some instances, s can equal x (from equations 1 and/or 2) and/or v can equal y (from equations 1 and/or 2). In other instances, constants/functions used to determine whether the control indicates can be different from constants/functions used to determine whether the target indicates. For example, x can equal 240 seconds, y can equal 2, s can equal 360 seconds, and v can equal 1.5. In addition, or alternatively, the windows over which the moving averages and standard deviations for the target and control can be the same or different.

In some instances, if the control indicates and the target does not indicate, a signal indicating a negative result can be sent, at 255 and/or the sample run can be terminated, at 260. In some embodiments, the negative result can be sent at 255 and/or the sample run can be terminated at 260 immediately (e.g., within 3 seconds) of the control indicating in the absence of the sample indicating. In other embodiments, upon the control indicating, the run can continue for a fixed period of time (e.g., 3 minutes, 5 minutes, or any other suitable time period) or for a dynamically determined period of time that is a function of, for example, time since the initiation of the run. If the target indicates during the period of time after the control indicates, a signal indicating a positive test result can be sent at 250 and/or the run can be terminated at 260. In yet other embodiments, upon the target indicating, the run can continue for a fixed period of time (e.g., 3 minutes, 5 minutes, or any other suitable time period) or for a dynamically determined period of time that is a function of, for example, time since the initiation of the run. If the control indicates during the period of time after the target indicates, a signal indicating a negative test result can be sent at 255 and/or the run can be terminated at 260. In instances in which more than one control is used, the absolute and/or relative timing at which each control and/or target indicates can be used to determine whether to send an indication of a positive result or a negative result.

In some embodiments, an indication of a positive test result and/or negative test result is ignored (e.g., not analyzed for, suppressed, not sent, reported, logged, and/or the basis for terminating a run) if it occurs in an initial portion of the analysis. In a LAMP analysis, a sample is typically inserted into a pre-heated heater-block. Typically, fluorophore characteristics cause the target and control signal to be weaker at lower temperatures (e.g., before the sample reaches thermal equilibrium with the heater-block). Such weaker signals may not reliable indications of sample positivity/negativity. In addition, the rate at which fluorophore intensity increases as the sample nears thermal equilibrium typically decreases. Similarly stated during an initial portion of the sample run when the sample is approaching thermal equilibrium with the heater block, the target and control signals are typically rising and concave-down. Thus, in some instances, positive test results and/or negative test results can be ignored if the target and/or control signals, respectively, have a positive slope and negative concavity. Slope and concavity measurements of the target and/or control signals can be based on a time-windowing queue, similar to the moving averages and moving standard deviations discussed above. Once a negative slope or positive concavity for the target and/or control signal is detected, indications for that signal may no longer be ignored. In other instances, positive test results and/or negative test results can be ignored for a predetermined fixed time period. For example, target indications can be ignored during the first 180, 240, 300, 360, 420 seconds, or any other suitable time period, of the run. As another example, control indications can be ignored during the first 630, 690, 750, 810, 870 seconds, or any other suitable time period.

FIG. 3 is an experimental data from a FLOS-LAMP analysis of an example sample. Line 310 represents a moving average of the intensity of fluorophore that is associated with a quantity of a target analyte. Lines 320 and 322 represent the moving average of the intensity of the fluorophore offset in time+/−a multiple of a standard deviation of the intensity of the fluorophore, respectively. In this instance, the offset is 240 seconds and the multiple of the standard deviation is 3. Therefore, line 320 represents μ_{L}(t−240)+3σ_{L}(t−240), and line 322 represents μ_{L}(t−240)−3σ_{L}(t−240). At approximately 3000 seconds from run initiation, line 310 and line 320 cross, such that μ_{L}(3000)>μ_{L}(3000-240)+3σ_{L}(3000-240), representing the target indicating. Thus, at approximately 3000 seconds, a signal indicating a positive result can be sent and, optionally, the run can be terminated. Alternatively, the run can proceed for an additional period of time to assure that the target continues to indicate.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of the embodiments as discussed above.

For example, although methods described herein generally relate to FLOS-LAMP analyses and are particularly well suited to SARS-CoV-2 detection, it should be understood that the techniques described herein can be applied to many other analyte and/or target detection schemes. Similarly stated, embodiments described herein are not limited to SARS-CoV-2 detection but can be applied to any analyte that can be selectively amplified or concentrated, through, for example, LAMP, polymerase chain reaction (PCR), chemical synthesis, electrochemistry, bioproduction, chromatography, electrophoresis, isoelectric focusing, gravimetric separation, etc. Although embodiments described herein generally describe fluorescent signals that are associated with or can be correlated to a quantity or concentration of an analyte, analytes can be detected by any suitable means such as, for example a pH-driven colorimetric signal from Real-Time Loop-Mediated Isothermal Amplification (RT-LAMP), or any other suitable colorimetric, electric, electro-chemical, optical absorbance, etc. signal. Similarly stated, the method shown and described with reference to FIG. 2 is well suited to any suitable analysis where a “positive” result is characterized by exponential or other rapid growth of a signal from a relatively low baseline.

In particular embodiments, the methods disclosed herein may be used to determine the presence or absence of an analyte by detecting and/or measuring a signal generated via PCR. A variety of different PCR methods may be used, including but not limited to: basic PCR, reverse transcriptase (RT)-PCR, Hot-start PCR, competitive PCR, or quantitative real-time (qRT)-PCR, e.g., as described at https://www.dot.promega.dot.com/resources/guides/nucleic-acid-analysis/per-amplification/ and references discussed therein.

In particular embodiments, the methods disclosed herein may be used to determine the presence or absence of an analyte by detecting and/or measuring a signal generated via isothermal nucleic acid amplification. Isothermal amplification of nucleic acids is an alternative to polymerase chain reaction (PCR). The advantage of these methods is that the nucleic acids amplification can be carried out at constant temperature, unlike PCR, which requires cyclic temperature changes. In certain embodiments, the isothermal nucleic acid amplification is performed using, e.g., loop mediated isothermal amplification (LAMP), nucleic acid sequence based amplification1 (NASBA), Helicase dependent amplification (HDA), Exponential amplification reaction of nucleic acids (EXPAR), Strand displacement amplification (SDA), Recombinase polymerase amplification (RPA), rolling circle amplification (RCA), e.g., as described in O. L. Bodulev1 and I. Yu. Sakharov, Biochemistry (Moscow), 2020, Vol. 85, No. 2, pp. 147166 and references cited therein, which is incorporated by reference herein in its entirety.

Parameters shown and described above with reference to FIG. 2 (e.g., the window for the moving average, the window for the standard deviation, the temporal offset (x), and the standard deviation multiple (y)), are generally described in the context of FLOS-LAMP and are selected based on the characteristic shape of positive target and/or control signals. A skilled data scientist, taking the above into account could readily select other appropriate parameters for signals having different characteristics.

In certain embodiments, the methods disclosed herein may be used to determine the presence of an analyte (e.g., (1) a detectable quantity and/or concentration and/or (2) or a quantity and/or concentration above a threshold) or the absence of an analyte (e.g., (1) the lack of a detectable quantity and/or concentration and/or (2) a quantity and/or concentration below a threshold). For example, in certain embodiments, the methods measure the presence or absence of a nucleic acid component of an analyte, e.g., using PCR, thus determining the presence or absence of the analyte in the sample. In particular embodiments, the analyte is an infectious agent or a pathogen, or a component thereof. In particular embodiments, the infectious agent or pathogen is a virus, a bacteria, or a fungus. In particular embodiments, the infectious agent is an influenza virus or a coronavirus, e.g., SARS-CoV-2. In some embodiments, methods disclosed herein are used to determine the presence of the infectious agent or pathogen by detecting presence of infectious agent DNA or RNA, e.g., in a sample. In some embodiments, the sample is a biological sample obtained from a subject diagnosed with the infection or considered to be at risk of having or developing the infection. In other embodiments, the sample is a food product or beverage product. In some embodiments, the sample is obtained from a surface, e.g., a food preparation surface, a food or beverage package surface, or a surface in a home, rental home, or hotel, such as but not limited to a kitchen counter surface, a bathroom counter surface, a toilet, shower, or bathtub surface, or a table or dresser surface.

In certain embodiments, the analyte is a virus or component thereof. In some embodiments, the sample is a biological sample obtained from a subject diagnosed with or suspected of being or at risk of being infected with the virus. In particular embodiments, the virus is a norovirus, rotavirus, adenovirus, astrovirus, influenza virus, coronavirus, parainfluenza virus, respiratory syncytial virus, human immunodeficiency virus (HIV), human T lymphotropic virus (HTLV), rhinovirus, hepatitis A virus, hepatitis B virus, Epstein Barr virus, or West Nile virus. In particular embodiments, the virus is SARS-CoV-2.

In certain embodiments, the virus is an influenza virus, including but not limited to any of the types or subtypes, lineages, or clades disclosed herein. There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). Current subtypes of influenza A viruses that routinely circulate in people include: A(H1N1) and A(H3N2). Certain circulating influenza A(H1N1) viruses are related to the pandemic 2009 H1N1 virus that emerged in the spring of 2009 and caused a flu pandemic. This virus, scientifically called the “A(H1N1)pdm09 virus,” and more generally called “2009 H1N1,” has continued to circulate seasonally since then. Influenza A(H3N2) viruses have formed many separate, genetically different clades in recent years that continue to co-circulate.

Influenza B viruses are not divided into subtypes, but instead are further classified into two lineages: B/Yamagata and B/Victoria.

In certain embodiments, the virus is a coronavirus, including but not limited to any of the types or subtypes or groupings disclosed herein. Coronaviruses are named for the crown-like spikes on their surface. There are four main sub-groupings of coronaviruses, known as alpha, beta, gamma, and delta. Seven coronaviruses that can infect people are: the common human coronaviruses: 229E (alpha coronavirus); NL63 (alpha coronavirus); OC43 (beta coronavirus); HKU1 (beta coronavirus); and other human coronaviruses: MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19. Humans commonly are infected with human coronaviruses 229E, NL63, OC43, and HKU1.

In certain embodiments, the virus is SARS-CoV-2. A new disease called coronavirus disease 2019 (COVID-19) has been reported. COVID-19 is caused by infection with the novel coronavirus, SARS-CoV-2 or 2019-nCoV. In some embodiments, the analyte is detected in a biological sample obtained from a subject diagnosed with or is considered at risk of having or developing COVID-19.

In certain embodiments, the analyte is a bacterium or component thereof. In some embodiments, the sample is a biological sample obtained from a subject diagnosed with or considered at risk of having a bacterial infection. In certain embodiments, the bacterium is one of the following: Acinetobacter, Bacteroides, Burkholderia, Clostridium, Enterobacteriaceae, Enterococcus, Klebsiella, Staphylococcus, Streptococcus, Morganela, Mycobacterium, Neisseria, Pseudomonas, or Stenotrophomonas, including any of the following: Acinetobacter baumannii, Bacteroides fragilis, Burkholderia cepacia, Clostridium difficile, Clostridium sordellii, Carbapenem-resistant Enterobacteriaceae), Enterococcus faecalis, Klebsiella pneumonia, Staphylococcus aureus, including Methicillin-resistant Staphylococcus aureus (MRSA) and Vancomyin-resistant Staphylococcus aureus), Morganella morganii, Mycobacterium abscessus, Psuedomonas aeruginosa, Stenotrophomonas maltophilia, Mycobacterium tuberculosis, Streptococcus pneumonia, Neisseria meningitidis, or Vancomycin-resistant Enterococci.

In certain embodiments, the analyte is a fungus. In particular embodiments, the sample is a biological sample obtained from a subject diagnosed with or is considered at risk of having or developing a fungal infection. In certain embodiments, the fungus is any of the following: Aspergillis, Candida (including Candida auris), Cryptococcus neoformans, Pneumocystis (including Pneumocystis jirovecii), Mucormycetes, Taloromyces, Candida, Blastomyces, Coccidioides, Histoplasma, Cryptococcus (including Cryptococcus gattii), or Paracoccidioides.

In addition, some methods described herein describe terminating a sample run when a target or control indicates (optionally, after a waiting period). It should be understood, however, that in other embodiments, the sample can be run (e.g., the target analyte can be selectively amplified) to a maximum duration (e.g., 60 minutes, 90 minutes, etc.). In such an embodiment, an indication of a positive result can be sent if the target has indicated in that time period (optionally accepting indications during an excluded initial period). An indication of a negative result can be sent if the control has indicated in the time period. In other scenarios, a signal indicating that the test has failed or is indeterminate can be sent.

Where methods and/or events described above indicate certain events and/or procedures occurring in a certain order, the ordering of certain events and/or procedures may be modified. Additionally, certain events and/or procedures may be performed concurrently in a parallel process when possible, as well as performed sequentially as described above.

What is claimed is:

1. A method, comprising:
depositing a sample in an instrument configured to selectively amplify an analyte;
receiving a plurality of signals, each signal from the plurality of signals associated with a quantity of an analyte at an instance of time;
calculating, for each instance of time, a moving average of the quantity of the analyte and a moving standard deviation of the quantity of the analyte based on a subset of the plurality of signals associated with a period of time ending at that instance of time; and
comparing the moving average of the quantity of the analyte at a first instance of time with a sum of (1) the moving average for a second instance of time and (2) a multiple of the moving standard deviation at the second instance of time, the second instance of time being an amount of time before the first instance of time.

2. The method of claim 1, wherein the sample is a biological sample.

3. The method of claim 2, wherein the biological sample is selected from the group consisting of: serum, blood, salivary secretions, lacrimal secretions, respiratory secretions, nasal fluid, a mucous sample, and intestinal secretions.

4. The method of claim 1, wherein the analyte is a polynucleotide sequence.

5. The method of claim 4, wherein the polynucleotide sequence is a polynucleotide sequence of a virus.

6. The method of claim 1, wherein:
the instrument is a FLOS-LAMP instrument; and
the analyte is a characteristic sequence of a SARS-Cov-2 genome.

7. The method of claim 1, further comprising:
sending a signal indicating a positive result based on the moving average of the quantity of the analyte at the first instance of time being greater than the sum of (1) the moving average for the second instance of time and (2) a multiple of the moving standard deviation at the second instance of time.

8. The method of claim 7, wherein the signal indicating that a positive result is obtained is not reported to a user if the signal was obtained within a predetermined time period after a start of the analyte being selectively amplified.

9. The method of claim 7, wherein the signal indicating that a positive result is obtained is not reported to a user while the quantity of the analyte as a function of time has a positive slope and a negative concavity within a predetermined time period after a start of the analyte being selectively amplified.

10. The method of claim 1, wherein an analysis of the analyte is terminated within a predetermined time of determining that the moving average of the quantity of the analyte at the first instance of time being greater than the sum of (1) the moving average for the second instance of time and (2) a multiple of the moving standard deviation at the second instance of time.

11. The method of claim 1, wherein the plurality of signals is a first plurality of signals, the method further comprising:
receiving a second plurality of signals, each signal from the second plurality of signals associated with a quantity of a control at an instance of time;
calculating, for each instance of time, a moving average of the quantity of the control and a moving standard deviation of the quantity of the control based on a subset of the second plurality of signals associated with a period of time ending at that instance of time; and
comparing the moving average of the quantity of the control at the first instance of time with a sum of (1) the moving average of the quantity of the control for a second instance of time and (2) a multiple of the moving standard deviation of the quantity of the control at the second instance of time, the second instance of time being an amount of time before the first instance of time; and
sending a signal indicating a negative result based on:
the moving average of the quantity of the control at the first instance of time being greater than the sum of (1) the moving average of the quantity of the control for the second instance of time and (2) a multiple of the moving standard deviation of the quantity of the control at the second instance of time, and
the moving average of the quantity of the analyte at the first instance of time being less than the sum of (1) the moving average of the quantity of the analyte for the second instance of time and (2) a multiple of the moving standard deviation of the quantity of the analyte at the second instance of time.

12. The method of claim 11, wherein the signal indicating that a negative result is obtained is not reported to a user if the signal was obtained within a predetermined time period after a start of the analyte being selectively amplified.

13. The method of claim 11, wherein the signal indicating that a negative result is obtained is not reported to a user while the quantity of the analyte as a function of time has a positive slope and a negative concavity within a predetermined time period after a start of the analyte being selectively amplified.

14. The method of claim 1, wherein the plurality of signals is a first plurality of signals, the method further comprising:
receiving a second plurality of signals, each signal from the second plurality of signals associated with a quantity of a control at an instance of time;
calculating, for each instance of time, a moving average of the quantity of the control and a moving standard deviation of the quantity of the control based on a subset of the second plurality of signals associated with a period of time ending at that instance of time; and
comparing the moving average of the quantity of the control at the first instance of time with a sum of (1) the moving average of the quantity of the control for a second instance of time and (2) a multiple of the moving standard deviation of the quantity of the control at the second instance of time, the second instance of time being an amount of time before the first instance of time; and
sending a signal indicating a positive result based on:
the moving average of the quantity of the control at the first instance of time being less than the sum of (1) the moving average of the quantity of the control for the second instance of time and (2) a multiple of the moving standard deviation of the quantity of the control at the second instance of time, and
the moving average of the quantity of the analyte at the first instance of time being greater than the sum of (1) the moving average of the quantity of the analyte for the second instance of time and (2) a multiple of the moving standard deviation of the quantity of the analyte at the second instance of time.

15. The method of claim 1, wherein the plurality of signals is a first plurality of signals, the method further comprising:
receiving a second plurality of signals, each signal from the second plurality of signals associated with a quantity of a control at an instance of time;
calculating, for each instance of time, a moving average of the quantity of the control and a moving standard deviation of the quantity of the control based on a subset of the second plurality of signals associated with a period of time ending at that instance of time; and
comparing the moving average of the quantity of the control at the first instance of time with a sum of (1) the moving average of the quantity of the control for a second instance of time and (2) a multiple of the moving standard deviation of the quantity of the control at the second instance of time, the second instance of time being an amount of time before the first instance of time; and
sending a signal indicating a positive result based on:
the moving average of the quantity of the control at the first instance of time being greater than the sum of (1) the moving average of the quantity of the control for the second instance of time and (2) a multiple of the moving standard deviation of the quantity of the control at the second instance of time, and
the moving average of the quantity of the analyte at a second instance of time being greater than the sum of (1) the moving average of the quantity of the analyte for the second instance of time and (2) a multiple of the moving standard deviation of the quantity of the analyte at the second instance of time, the second instance of time occurring within a predetermined period of time after the first instance of time.

16. The method of claim 1, wherein the period of time ending at the first instance of time is at least 20 seconds long.

17. The method of claim 1, wherein the period of time ending at the first instance of time is a predetermined constant length of time.

18. The method of claim 1, wherein a length of the period of time ending at the first instance of time is dynamically determined based on a function of elapsed time.

19. The method of claim 1, wherein the plurality of signals is at least one of a plurality of electrochemical signals or a plurality of fluorescent signals indicative of a quantity of a polynucleotide undergoing amplification.

20. The method of claim 1, wherein:
wherein the plurality of signals is indicative of a quantity of a polynucleotide undergoing an amplification reaction; and
a length of the period of time ending at the first instance of time is dynamically determined based on a function of a temperature of the amplification reaction.

21. The method of claim 1, wherein the second instance of time is at least 180 seconds before the first instance of time.

22. The method of claim 1, wherein the multiple of the moving standard deviation is at least 1.5.

23. The method of claim 1, wherein the multiple of the moving standard deviation is a predetermined constant.

24. The method of claim 1, wherein the multiple of the moving standard deviation is dynamically determined as a function of the moving average at the first instance of time.

25. The method of claim 1, wherein the plurality of signals is a first plurality of signals associated with the intensity of a first fluorophore, the method further comprising:
receiving a second plurality of signals, each signal from the second plurality of signals associated with an intensity of a second fluorophore;
calculating, for each instance of time, a moving average of the intensity of the second fluorophore and a moving standard deviation of the intensity of the second fluorophore based on a subset of the second plurality of signals associated with a period of time ending at that instance of time; and
comparing the moving average of the intensity of the second fluorophore at a first instance of time with a sum of (1) the moving average for a second instance of time and (2) a multiple of the moving standard deviation at the second instance of time, the second instance of time being an amount of time before the first instance of time.

26. The method of claim 1, wherein the plurality of signals is a first plurality of signals, the method further comprising:
receiving a second plurality of signals, each signal from the second plurality of signals associated with a quantity of RNaseP in the sample at an instance of time;
calculating, for each instance of time, a moving average of the quantity of RNaseP and a moving standard deviation of the quantity of RNaseP based on a subset of the second RNaseP plurality of signals associated with a period of time ending at that instance of time; and
comparing the moving average of the quantity of RNaseP at a first instance of time with a sum of (1) the moving average of the quantity of RNaseP for a second instance of time and (2) a multiple of the moving standard deviation of the quantity of RNaseP at the second instance of time, the second instance of time being an amount of time before the first selected instance of time; and
sending a signal indicating an insufficient volume of the sample based on:
the moving average of the quantity of RNaseP at the first instance of time being greater than the sum of (1) the moving average of the quantity of RNaseP for the second instance of time and (2) a multiple of the moving standard deviation of the quantity of RNaseP at the second instance of time, and
the moving average of the quantity of the analyte at the first instance of time being less than the sum of (1) the moving average of the quantity of the analyte for the second instance of time and (2) a multiple of the moving standard deviation of the quantity of the analyte at the second instance of time.

27. The system for analyzing a sample for the presence of an analyte, the system comprising:
a well configured to receive a reaction tube containing an analyte;
a light emitting source configured to emit an excitation light at a wavelength to illuminate the analyte in the reaction tube;
an optical detector configured to receive optical signals in response to the analyte being illuminated by the excitation light; and
a processor operably coupled to the light emitting source and the optical detector configured to:
activate the light emitting source;
receive a plurality of signals from the optical detector, each signal from the plurality of signals associated with the optical signals and indicative of a quantity of an analyte at an instance of time;
calculate, for each instance of time, a moving average of the quantity of the analyte and a moving standard deviation of the quantity of the analyte based on a subset of the plurality of signals associated with a period of time ending at that instance of time; and
compare the moving average of the quantity of the analyte at a first instance of time with a sum of (1) the moving average for a second instance of time and (2) a multiple of the moving standard deviation at the second instance of time, the second instance of time being an amount of time before the first instance of time.

28. A non-transitory computer-readable medium storing instructions configured to cause a processor to:
receive a plurality of signals, each signal from the plurality of signals associated with a quantity of an analyte at an instance of time;
calculate, for each instance of time, a moving average of the quantity of the analyte and a moving standard deviation of the quantity of the analyte based on a subset of the plurality of signals associated with a period of time ending at that instance of time; and
compare the moving average of the quantity of the analyte at a first instance of time with a sum of (1) the moving average for a second instance of time and (2) a multiple of the moving standard deviation at the second instance of time, the second instance of time being an amount of time before the first instance of time; and
send a signal indicating a positive result based on the moving average of the quantity of the analyte at the first instance of time being greater than the sum of (1) the moving average for the second instance of time and (2) a multiple of the moving standard deviation at the second instance of time.

29. A method of determining the presence or absence of an analyte is a biological sample, the method comprising
depositing a biological sample in an instrument configured to selectively amplify an analyte;
receiving a plurality of signals, each signal from the plurality of signals associated with a quantity of an analyte at an instance of time;
calculating, for each instance of time, a moving average of the quantity of the analyte and a moving standard deviation of the quantity of the analyte based on a subset of the plurality of signals associated with a period of time ending at that instance of time; and
comparing the moving average of the quantity of the analyte at a first instance of time with a sum of (1) the moving average for a second instance of time and (2) a multiple of the moving standard deviation at the second instance of time, the second instance of time being an amount of time before the first instance of time,
wherein the biological sample is determined to contain the analyte in a greater than a threshold quantity when the moving average of the quantity of the analyte at the first instance of time is greater than the sum of (1) the moving average for the second instance of time and (2) a multiple of the moving standard deviation at the second instance of time, and
wherein the biological sample is determined to contain less than a threshold quantity of the analyte when:
the moving average of the quantity of the control at the first instance of time is greater than the sum of (1) the moving average of the quantity of the control for the second instance of time and (2) a multiple of the moving standard deviation of the quantity of the control at the second instance of time, and
the moving average of the quantity of the analyte at the first instance of time is less than the sum of (1) the moving average of the quantity of the analyte for the second instance of time and (2) a multiple of the moving standard deviation of the quantity of the analyte at the second instance of time.

30. The method of claim 29, wherein the biological sample is selected from the group consisting of: serum, blood, salivary secretions, lacrimal secretions, respiratory secretions, nasal fluid, nasal swab, oral swab, a mucous sample, and intestinal secretions.

31. The method of claim 29, wherein the analyte is a polynucleotide sequence.

32. The method of claim 31, wherein the polynucleotide sequence is a polynucleotide sequence of a virus.

33. The method of claim 32, wherein the virus is a SARS-CoV2 vials or variant thereof.

34. The method of claim 29, wherein the instrument is a FLOS-LAMP instrument.

35. (canceled)
